(19) United States
(12) Patent Application Publication
(10) Pub. No.: US 2014/0266860 A1
(43) Pub. Date: Sep. 18, 2014

(54) Method and system for activity detection and classification

(71) Applicants: Gaddi BLUMROSEN, Tel-Aviv (IL); Ben FISHMAN, Zur-Igal (IL); Yosef YOVEL, Ramat Gan (IL)

(72) Inventors: Gaddi BLUMROSEN, Tel-Aviv (IL); Ben FISHMAN, Zur-Igal (IL); Yosef YOVEL, Ramat Gan (IL)

(21) Appl. No.: 14/210,972

(22) Filed: Mar. 14, 2014

Related U.S. Application Data
(60) Provisional application No. 61/785,700, filed on Mar. 14, 2013.

(51) Int. Cl. A61B 8/08; G01S 13/89; G01S 15/89; G01S 15/66; G01S 13/66

(52) U.S. Cl. CPC A61B 8/08; G01S 15/66; G01S 13/66; G01S 15/89; G01S 13/89
USPC 342/106; 367/87; 342/385; 367/89; 342/104; 367/7; 600/437

(57) ABSTRACT

A method for distinguishing a target, wherein the target includes one or more objects of interest possibly located among a plurality of objects. The method comprises the following stages: obtaining and processing sonar or radar raw data; tracking the objects of the plurality using the processed raw data; grouping the tracked objects by associating them into one or more groups, while hierarchically arranging the tracked objects in the groups and controllably applying prior knowledge at least about characteristic features and/or constraints of the target's class; classifying the groups to classes and determining whether any of the groups matches to the target's class.

### FIELD OF THE INVENTION

The present invention relates to the field of sonar or radar based techniques for detection of motion and other activity of human and non-human objects, for various applications such as security, detecting suspected activity in of land or underwater environments, health care, especially for monitoring of newborn as well as sick/elderly/handicapped persons, navigation in surroundings with reduced vision range, assistance to blind people, monitoring animals and birds, etc.

### BACKGROUND OF THE INVENTION

Systems designed for detecting, tracking and classification of motion can be sorted by their technology and the applied processing methods. The main technologies serving this purpose are video recording systems, active video systems, radar systems and sonar systems.

The use of a narrow band radar has been proposed by U.S. Pat. No. 7,924,212B2 and also in [1] for the detection and classification of patients' movements and location based on the Doppler effect. Motion kinematics, like walking speed and gait variability were acknowledged as features that can be used to assess severity of Parkinson Disease[2, 3].

An Ultra Wide-Band (UWB) radar, which uses a large portion of the radio spectrum, has recently been suggested for acquisition of motion kinematics [4]. The UWB radar can be used for applications like cardiac bio-mechanic assessment and chest movement assessment[5].

Radar-based systems suffer from some disadvantages like vast electromagnetic radiation, high cost, difficulty in differentiating between different body part movements, extensive computation resources.

Sonar systems, which utilize acoustic waves, may be used as an alternative to radar systems. Sonar systems are cheaper, less harmful, more ecologically clean, and less noticeable by persons/objects being tracked/observed, which is quite important for applications in the field of security and medical care.

U.S. Pat. No. 5,519,669A describes acoustic surveillance of objects and human traffic in a spatial zone of a financial transaction device, which is used to detect movement within the zone. Several specific types of detected movement defined as abnormal trigger an alert to a remote monitoring station. The alerts are automatically prioritized using rule-based criteria. Enhanced surveillance of the alert site by audio links as well as site alert history information are provided.

U.S. Pat. No. 3,681,745A describes an acoustic detection system of the Doppler variety, provided with digital filtering circuitry for eliminating the false alarming effects of various spurious sources which are situated in or near a space being monitored for a predetermined type of motion. Squaring circuitry is provided for converting the normally analog waveforms to digital waveforms whereby simple digital band-pass filters may be used to sharply discriminate against those frequencies considered non-attributable to the particular motion of interest.

US2003222778A proposes arranging overlapping range rings from a pair of non-scanning radar or sonar transducers, creating a grid structure within a surveillance area defined by their overlapping beam widths. Using PN coded transmission signals and Doppler signal processing, intruder targets are detected, located, and tracked as they move throughout the grid structure. Intruders are identified by comparing their movement pattern to those of known intruders. Three dimensional surveillance areas can be monitored using 3 or more transducer sites.

Advanced methods of distinguishing between different objects using sonar systems do not use sensors attached to objects being tracked; also, they utilize various regimes of sonar transmission and various processing techniques; the processing may be based on statistical/mathematical models, for example on algorithms for tracking or pattern recognition.

US2007121097A discloses a method and a system for range detection. The system may include a sensing unit for detecting a location and movement of a first object, and a processor for providing a measure of the movement. The processor can convert the measure to a coordinate signal for moving a second object in accordance with location and movement of the first object. The system can include a pulse shaper for producing a pulse shaped signal and a phase detector for identifying a movement from a reflected signal. A portion of the pulse shaped signal can be a frequency modulated region, a constant frequency region, or a chirp region. In one arrangement, the pulse shaper can be a cascade of all-pass filters for providing phase dispersion.

US2003055640A suggests a parameter estimator for estimating a set of parameters for pattern recognition; it has a recognizer for receiving a training set having members. The recognizer performs recognition on the members of the training set using a current set of parameters and based upon a predetermined group of elements. A set generator associated with the recognizer generates at least one equivalence set containing recognized members of the training set, which are used by a target function determiner associated with the set generator to calculate a target function using the set of parameters. A maximizer updates the parameter set so as to maximize the calculated target function. A speech recognized comprises a Viterbi recognizer. Acoustic modeler embeds acoustic constraints into a statistical model.

US2004042639A describes a technique for motion classification using dynamic 5 system models. Portions of an input measurement sequence are classified into a plurality of regimes by associating each of a plurality of dynamic models with one a switching state such that a model is selected when its associated switching state is true. In a Viterbi-based method, a state transition record is determined, based on the input sequence. A switching state sequence is determined by backtracking through the state transition record. Finally, portions of the input sequence are classified into different regimes, responsive to the switching state sequence. In a variation-based method, the switching state at a particular instance is also determined by a switching model. The dynamic model is then decoupled from the switching model. Parameters of the decoupled dynamic model are determined responsive to a switching state probability estimate. A state of the decoupled dynamic model corresponding to a measurement at the particular instance is estimated, responsive to the input sequence. Parameters of the decoupled switching model are then determined responsive to the dynamic state estimate. A probability is estimated for each possible switching state of the decoupled switching model. A switching state sequence is determined based on the estimated switching state probabilities. Finally, portions of the input sequence are classified into different regimes, responsive to the determined switching state sequence. In one embodiment, one or more constraints can be imposed on the classification.

US2012106298A discloses a gesture recognition apparatus and method. The gesture recognition apparatus includes an ultrasound transmitter, an ultrasound receiver, a dividing module, a computing module, a gesture library, and a recognition module. The dividing module is configured to divide reflected ultrasound signals into a plurality of frames according to time intervals. The computing module is configured to obtain an eigenvalue of each frame. The classifying module is configured to filter the eigenvalues to obtain gesture eigenvalues, and to obtain a matrix of probabilities of the gesture eigenvalues. The recognition module is configured to search reference matrices of probabilities from the gesture library for matching with the matrix of probabilities, and to recognize the gesture eigenvalues as a reference gesture corresponding to the reference matrix of probabilities if the reference matrix of probabilities is found.

There is still a need in such a technology which would provide a system with effective, controllable processing not requiring prior training of the system, without placement of active markers or inertial sensors at objects, and which would allow providing not only effective differentiation between various objects (say, static and dynamic, human and non-human), but also distinguishing between details/body parts of a specific object.

### OBJECT AND SUMMARY OF THE INVENTION

It is therefore an object of the present invention to provide a technique that allows satisfying the above requirements.

The technique of interest should be relatively simple, controllable, capable of assessing objects for differentiation and tracking thereof, and should be capable of further classifying the objects into clusters of dynamic objects and clutter.

The Inventors have realized that, in order to achieve accurate results, all presently known techniques for motion assessment utilize very complex, time and resource consuming processing methods.

The Inventors have developed their own, relatively simple and inexpensive though effective method applicable for processing echoes obtained from objects being watched by sonar or radar systems. The Inventors' two presently unpublished articles [6, 7] are incorporated herein by reference in their entirety.

According to a first aspect of the invention, there is proposed a method for distinguishing/acquisition of motion or activity among a plurality of objects possibly including one or more targets.

The plurality of objects may include static and dynamic, human and non-human objects. In the frame of the present patent application, the term target should be understood as object(s) of interest. The term target is accepted in sonar and radar systems as a group/assembly comprising one or more somehow associated objects.

The class of target can be, for example: static, dynamic, human, non-human; the class can be complex such as “dynamic non-human”, etc. and specified to types, for example a dynamic non-human target may be an animal or a mechanism; a human dynamic target may be an elderly person, a baby, a sportsman, a handicapped person, an intruder, etc.

Targets may be combined, i.e., for example may comprise an assembly of objects such as a human body having a static torso and moving body parts, with a static bag.

The method is controllable and may be adapted to perform effective acquisition and classification of motions/activity of various objects, but especially of dynamic objects of interest, such as animals, robots, humans, as well as effective distinguishing of types of activities of humans of various ages and at various circumstances (presented by various implementations of the method).

The method may therefore be formulated as follows.

A method for distinguishing/recognizing a target (any target naturally belonging to a specific class), the target including one or more objects of interest possibly located among a plurality of objects,

the method comprising stages of:

obtaining and processing sonar or radar raw data (being data about sonar or radar echoes),

tracking the objects of the plurality using the processed raw data,

grouping the tracked objects by associating them into one or more groups, while hierarchically arranging the tracked objects in the groups and controllably applying prior knowledge at least about characteristic features or constraints of the target's class,

classifying the groups to classes, and determining whether any of the groups matches at least to the target's class (thereby distinguishing/recognizing said to target if located among the plurality of objects).

It goes without saying that, for obtaining sonar or radar raw data, the plurality of objects should be exposed to sonar or radar signals, and echoes from the objects should be received; however the site and time where such takes place may coincide or not coincide with the site and time of processing the sonar or radar echoes comprising raw data.

Class(es) of possible/expected target(s) and their characteristic features and/or constraints may be known in advance, regardless whether a specific target is known in advance. The proposed method does not require prior knowledge about features/constraints of the specific target.

The target may be distinguished as one of the formed groups, upon classifying the groups. No prior training is required.

The method may handle more than one target, in this case the prior knowledge may comprise characteristic features of classes of all expected targets.

The controlled use of characteristic features of the target's class as prior knowledge (or constraints) at the grouping stage allows reducing complexity of computation at the classification stage and increasing the accuracy of motion acquisition.

The controlled use of such prior knowledge means, inter alia, that the constraints for static and/or dynamic objects may be selected according to a specific target and specific implementation of the method. For example, the constraints may include sonar/radar signatures of dynamic objects, but may also include just reflection structure according to which dynamic objects such as humans may be distinguished from walls or other static objects. Other prior knowledge constraints for the grouping stage will be discussed in more details further in the summary and in the detailed description.

As mentioned, the targets may be different. The targets may be static, for example at indoor environment a table, a chair, a wall, and at outdoor environment, of land or underwater, rocks, etc. The targets may be dynamic or static-dynamic, and of different types, for example the class of human targets may have various types associated with a specific age and condition (for instance handicapped, elderly, babies, in a bed or moving), the class of non-human dynamic targets may comprise types such as animals, robots, cars, and other devices.

The prior knowledge may also comprise characteristics and/or constraints of the target's type, of the medium (for example air or water) of the environment, etc.

Implementations of the method may be of various types depending on the purpose (security, medical care, underwater monitoring, etc.), which will be described later.

Therefore, the prior knowledge may also comprise characteristics and/or constraints of a specific implementation type, for example: characteristics of a specific medium, and dimension ranges, velocity ranges, typical and atypical acceleration ranges per group of objects, and per object in the group.

The method may further comprise controllably applying prior information of at least the target's class characterizing features and/or constraints at the stage of tracking; it should be noted that the prior information applied at the stage of tracking may differ from the prior knowledge (constraints) applied at the stage of grouping.

The method preferably comprises utilizing the sonar or the radar signals having high bandwidth, which allows increasing accuracy and resolution of necessary measurements.

In the definition given below, the term “signal” should be understood as a sonar or a radar signal.

The above-mentioned method may comprise a preliminary stage of
receiving echoes of the signals from the objects, deriving the raw data from said echoes, and forwarding the raw data for processing (which may be performed at a different site).

More specifically, the method may further comprise:
performing the raw data processing, thereby obtaining one or more echo properties taken for an echo “k” at time instance “m”, the one or more echo properties being selected from an echo properties list comprising at least τₖ^{m}, Iₖ^{m}, ρ_{k,l}^{m}, ρₖ^{m,m+1}
where
   τₖ^{m} is k'th echo's delay at time instance m
   Iₖ^{m} is k'th echo intensity at time instance m
   ρ_{k,l}^{m} is the cross-correlation coefficient between the k, and l echoes' shapes, at time instance m
   ρₖ^{m,m+1} is the auto-correlation coefficient between the k'th echoes' shapes, at time instances m, and m+1.
performing the stage of tracking the objects, comprising controlled processing of the one or more obtained echo properties and mapping thereof to objects, thereby obtaining, for each of the objects, one or more object properties taken for object n at time instance m, the one or more object properties being selected from an object properties list comprising at least
(dₙ^{m}, νₙ^{m}, Sₙ^{m}, Pₙ^{m}), where
   dₙ^{m}—is the n'th object location estimate at time instance m;
   νₙ^{m}—is the n'th object velocity estimate at time instance m, derived by either deviation of location estimates, dₙ^{m}, or by the Doppler effect;
   Sₙ^{m}—is the n'th object size estimate at time instance m, estimated for example by the number of echoes related to object n and their intensity
   Pₙ^{m}—is the n'th object pattern, for example estimated using spatial-temporal autocorrelation between echoes associated with object n;
performing the stage of grouping of the objects by controllably associating the objects into groups based on one or more similar said object properties, with the hierarchically arrangement of the objects being members in the groups so that each of the groups comprises a main object and at least one sub-object, thereby obtaining for each of the groups a combined set of object properties of all members of the i'th group, at time instance m,
   {dₙ^{m,G}^{ᵢ}, νₙ^{m,G}^{ᵢ}, Sₙ^{m,G}^{ᵢ}, Pₙ^{m,G}^{ᵢ} . . . dₙ₊₁^{m,G}^{ᵢ} . . . dₙ₊₂^{m,G}^{ᵢ}};
based on the combined set of object properties and their hierarchy, deriving one or more group features, for each i'th group over a time window “W”, the group features being selected from a group features list comprising at least ν^{Gi}, σ^{Gi}, and N^{Gi}, μ_{ρ}^{Gi}, σ_{ρ}^{Gi}, where
   ν^{Gi}—is average velocity in the group over a time window W; for instance, velocities may be relative to that of one moving or static object in the i'th group,
   σ^{Gi}—is average location standard deviation over the time window W, possibly relative to one static or moving object in the i'th group,
   N^{Gi}—is the number of dynamic/static objects in the i'th group over a time window W
   μ_{ρ}^{Gi}—is average auto-correlation of the objects in the i'th group
   σ_{ρ}^{Gi}—is standard deviation of the auto-correlation of the objects in the i'th group;
performing the classification stage by controllably applying, to the group features per group, at least prior knowledge on characterizing features and/or constraints of the targets' class, corresponding to one or more of the group features, thereby determining whether at least one of the groups matches to the target's class.

Preferably, the classification stage comprises obtaining a list of classes of the groups, determining class and type of each group, as well as level and type of activity at least of some of the groups.

It should be kept in mind that more echo's properties can be extracted from the raw data (for example, distortion of the echo pulse shape, etc.).

It should also be noted that all the properties and features discussed in the method are functions in the coordinates of time and space. The time coordinate is expressed by the index “m”, while the space coordinate is reflected by changes in delay, intensity, correlation, location, etc.

Additional group features may be derived according to the distribution of displacement, or velocity in the group, and spatial-temporal correlation properties between the objects in the group.

As mentioned before, the human motion tracking and classification method/system may be adapted to operate in different environments and mediums. One exemplary type of environment is any on-land environment, and another exemplary type is any underwater environment. Similar analysis tools can be applied to all of these environments, and be adapted to separate between human and non-human objects, and further, to classify human activity into different classes.

The proposed patent concept can be applied, in addition to on-land applications such as security, bio-medical implementations etc. also for s-called underwater applications (such as security, identifying humans in the water, distinguishing between divers and fish, etc).

The underwater security applications may include, for example marine applications to identify terror attacks, and to provide alerts based on detecting suspicious movements in the sea, near secured places and facilities.

The patent concept can also be applied to identify drowning people in swimming pools and in natural water reservoirs (lake, river, sea, ocean). By applying the described method, the operator/program will be able to identify whether a rock, a fish, a diver is/are detected, and whether the diver is swimming, standing, or performing another type of activity.

Further, more details about the stages will be disclosed.

The stage of tracking may be performed flexibly, wherein the controlled processing comprises

applying prior information about objects of interest, and

performing splitting and/or merging of the echoes for tracking newly appearing, disappearing, and/or transforming objects in the plurality.

The prior information for controlling the tracking stage may, for example, include accuracy degrees and sensitivity thresholds, and info about static or dynamic objects of interest (targets), for example in case the target is a human, the info may relate to typical or expected human activity, boundaries on ranges of velocities of different body parts. The prior information may further comprise a constraint of continuity or similarity of movement features, over time, of various body parts.

The tracking stage is preferably performed as a multi-object, statistical similarity tracking procedure, regardless any differences between the tracked objects, i.e. without attempts of pre-classifying thereof at the tracking stage.

The multi object tracking procedure may be based on various statistical criterions, such as Maximum Likelihood Estimator (MLE), Minimal Mean Square Error (MMSE).

The Inventors have proposed a simplified effective correlation tool for the statistic tracking procedure, being a simplified Branch Metric Approximation, wherein the branch metric M_{k,l}^{m} between echoes k and l for time instance m, can be defined essentially close to:

M_{k,l}^{m}=e^{-aΔD}^{_{k,l}}^{^{m}}(I_{k,l}^{m})^{β}(ρ_{k,l}^{m})^{γ}, (1)

and where

$$Δ {d}_{k , l}^{m} = \vert {d}_{k}^{m} - {d}_{l}^{m - 1} \vert , {I}_{k , l}^{m} = \frac{min ( {I}_{k}^{m} , {I}_{l}^{m - 1} )}{max ( {I}_{k}^{m} , {I}_{l}^{m - 1} )} ,$$

ρ_{k,l}^{m}, are measures of distance, intensity, and cross-correlation between the k'th and the l'th echoes (supposed objects), and
α, β, γ, are constants; may be determined experimentally and reflecting reliability and significance of the distance (d), the intensity (I), and the cross-correlation (ρ) measures respectively to the detection probability.

The prior information for controlling the tracking stage may therefore also comprise selecting the constants α, β, γ according to the specific objects of interest. It should be noted here, that the tracking stage operates on the echoes properties with their indexes (as also seen in the metric M_{k,l}^{m} above), and after merging, splitting, and deletion operations of the tracking, the output of the tracking stage presents the tracked objects, with their properties and related indexes.

The grouping stage of the method will be now disclosed in more details.

The stage of grouping the objects may further comprise
receiving object properties of each of the objects, upon being determined at the tracking stage;
associating the objects into groups by utilizing the received object properties, so that each of the groups is formed based on statistical similarity of at least one of the object properties for members of the group; thereby presenting each group as a combined set of object properties of all members of the group;
simultaneously with or after the association of the objects into groups, hierarchically arranging the objects in each of the groups by controllably applying the prior knowledge about at least the target's class (say, about dynamic objects of interest) in the form of one or more characteristic features or constraints selected from the list comprising at least dimensions, sonar/radar signatures and velocity ranges characteristic for the target's class (and possibly type: for example for the dynamic human objects).
   Therefore, additional control in the grouping stage (over the controlled applying of prior knowledge on class of the target) may be effected, for example, by selecting the object property for forming groups (for example location or velocity maybe used for integration (grouping) of multiple sonar/radar nodes), and/or by selecting the prior knowledge in the form of constraints related to types of objects of interest and according to specific implementations of the method, including of-land and underwater implementations

The constraints can be defined for each time instance m, or for a window of time W, and may comprise: specific body dimensions (usually proportional to intensity of the sum of objects that relate to the body), pattern of change and kinematic features of the class/type of target, like acceleration or velocity range.

Various types of implementations of the method may, for example, be intended for:
watching some static environment and expecting suspicious movement of targets which should be all static, for example for detecting burglary in a closed museum;
watching a baby or a sick patient in a bed, where weak movements are to be detected and analyzed,
watching an elderly person who may suddenly need help,
watching a number of people in a closed space, for example in a bank, and detecting any of these human targets approaching a static target, such as a safe or another human target such as a cashier;
monitoring of internal body parts for various medical or scientific purposes,
monitoring a human being which may appear in any underwater environment, for example a swimmer/diver/drowning person in a swimming pool or in natural water reservoirs, for example near some specific objects of civil or military interest in a sea/ocean;
   etc.

In different implementations, different displacement ranges, velocity ranges and/or acceleration ranges may serve as respective constraints, for example:

a) monitoring of sick patients or babies in a bed is associated with specific small ranges of displacement and velocity,
b) monitoring of intruders requires specific different velocity ranges, usually along with a constraint of conventional human body dimensions,
c) monitoring of children is usually performed with a constraint of childish body dimensions,
d) monitoring static objects which may be stolen/displaced and thus become quasi-dynamic, may be possible with a constraint of atypical acceleration,
e) monitoring elderly people which may fall may also be performed with a constraint of atypical acceleration,
f) monitoring internal body parts of humans/animals may require more than one sonar sensors/systems, dimension constraints of a different, suitable scale and accuracy which would allow further creation of medical images.
g) monitoring a human in the underwater environment may require additional information about specific different velocity ranges and characteristic movements of a swimming/diving/drowning person, about dynamic characteristics of possible underwater objects or clutter of objects including rocks, sand, flora and fauna, and fish, for further comparison and filtering them out; also, and of course about specific characteristics of the medium itself and the influence of that medium on the parameters used in the method.

For the hierarchical arrangement, the objects in each group may be sorted according to their different properties, for example according to objects' size (corresponding to intensity), so that the largest/most intensive object will be assigned as the main object and others as sub-objects. Another example is using a feature of relative location of the objects in the group which allows selecting the main object as that to which members of the group are closer than to other objects.

To indicate the arranged hierarchical order, the list of object properties may comprise additional mark/indication, for example specifically marking the main object's object properties.

It is to be emphasized that the above-proposed hierarchical arrangement of objects in the groups contributes to reduction of computational complexity of classification at a further stage.

As mentioned above, the hierarchical arrangement of the objects within the groups may be further followed by calculating the group features for each of the groups, based on the combined set of object properties and the hierarchy of members of the group, and providing the set of features, per group, to the classification stage.

The grouping stage may comprise iterative procedures of merging and/or splitting the formed groups, to adjust the obtained results of grouping. To adjust the grouping results, adjustment of the prior knowledge constraints may be required, for example by feedback.

The step of calculating the group features may form part of either the grouping stage, or the classification stage.

The grouping operation proposed by the Inventors, comprising the hierarchical arrangement of objects within the group using constraints related to the objects of interest, and being followed by further calculation of the group features, allows minimizing the computational complexity at the classification stage, and enables classifying the entire plurality of objects independently and effectively. The method is most advantageous in the cases when the objects include dynamic targets.

In other words, at the classification stage, the group features ensure effective distinguishing of the target as one (sometimes one or more) of the formed groups, if the target is present among the plurality of objects. The method also allows further acquisition of motion and activity of the target (if the target is dynamic), by applying further suitable classification.

Moreover, the described grouping stage (especially in combination with the preferred tracking stage and the classifying stage as further described) allows obtaining a simple and more controllable processing algorithm that minimizes the need in knowledge on exact object properties in advance, and excludes a tedious prior training phase from the method.

The classification stage of the proposed method may be performed as a multi-level classification procedure to distinguish static, dynamic, human, non-human targets and types and levels of the targets activity.

The classification stage comprises:
obtaining the group features derived for each of the groups, and
based at least on a set of the target's class features known in advance and respectively corresponding to the obtained group features, determining at least one group corresponding to the target's class (if the target is indeed present), thereby distinguishing the target.

It is logical that the prior knowledge of the classification stage comprises suitable information on other classes, so a list of classes comprising more than one class may be formed for the groups, the class of each group may be determined, wherein the classes comprising at least static/dynamic and human/non-human classes. Further, the activity type and level of groups may be classified.

The mentioned, known in advance set of target's class features respectively corresponding to the group features may, for example, comprise: velocity, standard deviation of location, spatial and temporal correlation and auto-correlation of echoes.

The set of the above-mentioned target features may be utilized (alternatively or in addition) in the form of criteria such as thresholds or ranges, produced from the target features, thus enabling distinguishing between static and dynamic, human and non-human groups (and therefore—targets).

It is understood that the final classification will depend also on the prior information and the prior knowledge selected and applied in the method at the preceding stages of processing.

The list of classes may be determined by a direct or a multi-level classifier.

The multi-level classification may be implemented, for example, by a k-NN classifier.

In the proposed classification, the activity type and level being defined at least for the human class.

The activity type and level may also be defined for animal targets.

The multi-level classification may optionally utilize sonar/radar signatures for classifying specific targets.

In one specific version of the method, the signals are sonar signals and the system implementing the method is a sonar system.

One important feature of the proposed method is utilizing a high bandwidth (wideband) sonar or radar signal. The wide range of frequencies of such a signal can give frequency related information about different objects' structure, material, and can also give accurate information about location of the objects.

In one version of the method, it comprises:
emitting a combination of sonar signals comprising one or more different frequencies, typically in a bandwidth between of about 5 kHz and of about 1000 kHz;
applying the combination of the sonar signals to environment where the plurality of objects possibly including one or more of targets, are expected to be located.

The method may further comprise collecting and processing echoes resulting from the combination of sonar signals, including:
processing the collected echoes by utilizing a set of a-priori knowledge thereby transforming the set to constraints for further use in the method; results of the processing thereby enabling more effective distinguishing of different objects of the plurality from one another.

The method therefore allows obtaining much more (additional) working features/parameters/properties of echoes, than other methods, therefore increasing at least the tracking, and the classification accuracy of the method.

The additional information may be then transformed into object properties which may then participate in forming the group features, thus may be helpful for any stage of the method—tracking, grouping, specification. For example, such features may be helpful for distinguishing the human objects from other objects, for distinguishing different human objects from one another and different body parts of one human object.

In one version of the method, such additional parameters may be directly used for distinguishing different parts of a dynamic object (such as body parts of a human object), at any stage of the method.

The mentioned combination of sonar signals may include one or more of those mentioned in the following non-exhaustive list:
a sonar signal comprising chirps, wherein the chirp being an acoustic pulse comprising pulse portions having different frequencies, for example a Frequency Modulated (FM) chirp;
a broad spectrum sonar signal comprising a sequence of short pulses having high power at a number of different frequencies;
a sonar signal being a combination of constant frequency (CW) portions and chirp (FM) portions;
a sonar signal comprising multiple harmonics;
sonar beams adaptable by at least one parameter from a non-exhaustive list comprising frequency, direction, width;
a combination of sonar signals emitted by more than one sonar transmitters, (say, for scanning a closed space in 2D or 3D constellation).

The high, actually ultrasonic, bandwidth and quite high energy of the sonar pulse (and/or high Signal to Noise Ratio SNR) enables distinguishing between objects using the enhanced, due to the high bandwidth, correlation properties/features, compared to narrow bandwidth techniques, and enables more accurate range assessment.

Finally, the method may also comprise creating an image of the target and preferably, also of its surroundings. The imaging may be based on utilizing the object properties obtained after the tracking stage, and/or on utilizing the group features obtained when performing the grouping (clustering) stage, monitored in the time-space coordinates. For example, the object property of location over time and group features such as average velocity or deviation may be utilized for creating a time-space diagram of each object, a time-space diagram of the group and there-from a general image of the target with specified portions of the image.

To increase the image resolution, a combination of sonar beams may be used. For example, a number of beams may be emitted by a number of sonar transmitters and received by a number of sonar sensors; alternatively, one or more sonar beams may be controllably directed.

The method may be further adapted for monitoring internal body parts of humans or animals, so as to create a medical image.

In another specific version, the method may be adapted for monitoring objects and distinguishing targets in an underwater environment.

According to a second aspect of the invention, there is also provided a suitable system for implementing the above-defined method.

In other words, there is provided a system designed for distinguishing (detecting, classifying and recognizing) a target, the target including one or more objects of interest possibly located among a plurality of objects exposed to sonar or radar signals,

the system comprising a processing block accommodating therein:

a unit for processing sonar or radar raw data obtained from the plurality of objects,

a unit for tracking the objects using the processed raw data,

a unit for grouping the tracked objects by associating the objects into groups while hierarchically arranging the tracked objects in the groups, wherein the unit for grouping is controlled at least by applying prior knowledge about characteristic features or constraints of the target's class;

a unit for classifying the groups and recognizing the target by matching the groups classes to the target's class.

The prior knowledge may also comprise characteristic features and/or constraints of the target's type. Further, the prior knowledge may comprise characteristic features and/or constraints of a specific implementation type, of the specific environment where the target is monitored (for example, the medium of the environment). Therefore, the target distinguishing becomes fine-tuned.

The system may be designed so that to accept prior knowledge with similar data about multiple targets and perform tracking, grouping and classifying of multiple targets in the most effective manner.

The system may be located remotely from, or directly at the site where the objects are watched by sonar or radar signals. The system may be in the form of the processing block which, for example, may be accommodated in the receiver of sonar or radar signals. In any case, the extended system may comprise the sonar/radar transceiver per se, i.e. a transmitter for transmitting the sonar or radar signals, a receiver for detecting echoes thereof, extracting raw data from the echoes, a synchronizing means between the receiver and the transmitter, and a communication line for forwarding the raw data to the processing block.

The system may further comprise multiple sonar/radar transmitters and a common receiver or an assembly of interconnected receivers capable of producing information for combined processing.

In a specific embodiment, the proposed system may comprise a sonar or a radar assembly configured for creating an image of the target, based on results produced by the mentioned processing block (i.e., that capable of implementing the proposed method).

The sonar assembly configured for creating images may, for example, constitute an ultrasound device for monitoring internal body parts (of any type existing in the market of medical devices). Accuracy of such a device may be improved by implementing the proposed technology for distinguishing targets (i.e., by utilizing the data supplied by the described processing block).

The sonar assembly configured for this purpose may be attachable to a patient's body (for example, a human's body).

In another specific embodiment, the system may be designed/configured for monitoring objects and distinguishing targets in an underwater environment.

As a third aspect of the invention, there is further provided a software product comprising computer implementable instructions and/or data for carrying out the above-described method, the software product being stored on an appropriate computer readable storage medium so that the software is capable of enabling operations of said method when used in a computerized system.

Additionally, there is provided a computer readable storage medium (a server, a hard disc, a removable disc, etc.) accommodating the software product or a portion thereof.

The method, the system, as well as the processing functionality thereof will be disclosed in more details, with reference to the following drawings, in the detailed description.

### BRIEF DESCRIPTION OF THE DRAWINGS

The patent or application file contains at least one drawing executed in color. Copies of this patent or patent application publication with color drawings will be provided by the Office upon request and payment of the necessary fee.

The invention will be further described with reference to the following non-limiting drawings in which:

FIG. 1a (prior art) schematically shows a simplified 1-D embodiment of an active sonar system.

FIG. 1b (prior art), schematically illustrates how a received sonar signal is periodically sampled in a receiver.

FIG. 1c (prior art), shows some sonar echoes/reflections received from objects in the medium and extracted at the sonar receiver.

FIG. 2 schematically shows a simplified flow-chart of a processing phase of the proposed method of motion acquisition, which comprises stages of tracking, grouping and classifying objects.

FIGS. 3a, 3b schematically illustrate an approach used by the Inventors for the statistical processing while tracking of the detected objects.

FIGS. 4a, 4b, 4c, 4d, 4e, 4f show experimentally obtained sonar diagrams which illustrate forming a small number of groups based on object properties of a much greater number of tracked objects.

FIGS. 5a, 5b, 5c, 5d show graphs which schematically illustrate forming of groups and selecting main elements in the groups.

FIG. 6 shows a schematic diagram for the optional categorizing of objects in the group to static and dynamic.

FIGS. 7a, 7b, 7c, 7d show experimental graphs illustrating results of using prior knowledge constraints at the grouping stage.

FIG. 8 depicts a two-level decision tree classifier for distinguishing classes of groups, and further activity type and activity level estimation.

FIG. 9 depicts an exemplary graphical diagram illustrating results of classification of multiple objects comprising a number of dynamic objects.

### DETAILED DESCRIPTION OF PREFERRED EMBODIMENTS

The examples presented below mainly relate to a sonar system, while are applicable, mutatis mutandis, to radar systems.

FIG. 1a schematically shows a simplified 1-D embodiment of an active sonar system having a transmitter 10, a receiver 12 synchronized by cable 16, and a processing unit 14 connected with the receiver (and optionally with the transmitter) via a communication line 18. The processing unit 14 of the prior art system has been modified by the Inventors and actually constitutes the main portion of the system implementing the proposed method. In a specific embodiment, the processing unit may be incorporated in the receiver 12. The flow chart of the proposed method will be described with reference to FIG. 2.

FIG. 1b (the upper diagram) schematically illustrates how a received sonar signal is periodically sampled in a receiver. The dashed vertical lines are time moments/instances (m−l, m, m+1 . . . ) of sampling sonar pulses emitted by the transmitter 10. Echoes appearing within the period T of pulse repetition are echoes of different objects, arriving to the sonar receiver 12 with respective different delays t, count from a sampling pulse of the transmitter.

FIG. 1c (the lower diagram) shows the enlarged view of the encircled portion of FIG. 1b (between a pulse repetition period), with the sonar echoes received from objects in the medium and extracted at the sonar receiver. The image of FIG. 1c shows extraction of echoes from the continuous received signal. The signal includes two main echoes in the period of the m'th pulse repetition. Maximal detected intensities of the two main echoes (shown by two vertical arrows) allow concluding that two objects are situated in the medium/area monitored by the sonar system.

The following description to FIGS. 1b and 1c mainly comprises the background information concerning sonar systems.

The simplest active sonar system is an active sonar node composed of an acoustic transmitter (speaker) 10, an acoustic receiver (microphone) 12, and a processing and storage unit 14. A pulse is transmitted into the medium where the desired object (say, a human) is located. The sonar receiver receives acoustical reflections (echoes) of the transmitted pulse from the medium. The reflections convey information about object location, structure, and sometimes composition. Each echo is characterized by its attenuation and a delay. The received signal at continuous time t and at the time instance m of pulse repetition is:

r(t)=r₀(Ø)Σ_{m,k}β_{m,k}(t−mT)p(t−mT−τ_{m,k})+n(t), (2)

where p(t) is a transmitted pulse implemented by Linear Frequency Modulated (FM) chirp, m is the sampling pulse index and T is the pulse repetition time, τ_{m,k} is the k'th echo delay in the m'th pulse, β_{m,k} is its related attenuation factor which is commonly assumed to be constant during the observation time and affected by geometrical factor and atmospheric attenuation factor due to humidity, r₀(ø) is an attenuation factor determined by the bearing angle ø of the sonar and by the sonar received and transmitted radiation pattern, and n(t) is an additive noise component. The noise includes thermal and amplifier noise which can be modeled by white Gaussian processes, distortion from non-linearity of the speaker membrane, and interference of other low frequencies echoes.

The sonar system can be extended to a set of multiple sensor nodes. Each sonar node is capable of sensing motion features in one dimension (1-D). To assess motion in three dimensions (3-D) at least three sensor nodes employed in different locations are needed.

Each object in the environment reflects the signal according to its so-called cross-section. The cross section depends on the object material, its surface, its size, and also depends on the transmitted pulse central frequency and bandwidth (thus allowing to extract additional parameters at different frequencies). The reflection coefficient depends on the acoustic wave frequency, and moves between 0 (absorption in the object) to 1 (full reflection). For pulse spectrums with frequencies in the range between 40-80 KHz (wave lengths values, λ, move between 0.68-1.37 mm) the body parts and large scatterers/reflectors in the medium, like walls, will reflect most of the transmitted pulse in a similar manner. Reflections from small body parts with surface of few centimeters, or a textured surface with different distances from the sonar, will have a varying pattern, which can be significantly different from those produced by wide reflectors like walls.

Implementations for transmitting and detecting of reflected sonar signals will not be discussed in the frame of the present patent application, since they may be performed by means known to those skilled in the art.

The received signals (in the receiver 12) are usually sampled every period of τₛ seconds. The samples of M consecutive pulse repetitions are stored in an observation matrix r of size M×Nₕ, where Nₕ=T/Tₛ is the number of samples in each pulse repetition. The received echoes are related to different reflections of the pulse at different locations in the medium, and therefore are related to the spatial dimension/coordinate. The distance between the reflecting object and the acquisition system is proportional to the echo's delay and to the propagation velocity which equals to the speed of sound. The pulse repetition period T includes signal reflections from all over the medium. From the raw data of the matrix “r”, the echoes properties are further calculated.

In the inventive technique which will be described below beginning from FIG. 2, the Inventors also propose using the high bandwidth sonar/radar signal. This allows receiving better resolution and accuracy in the measurements. Moreover, the reflection coefficients of objects to different frequencies of the sonar signal will be different and therefore—more information will be available (per desired frequencies up to 1000 kHz) for characterizing properties of different targets.

FIG. 2 schematically illustrates an exemplary flow-chart diagram of a so-called processing phase of the proposed method for motion acquisition and targets recognition. The processing phase comprises the following main stages: stage 19 of obtaining echo properties, stage 20 of tracking the objects, stage 22 of grouping the objects, and a classifying stage 24 (shown by the respective blocks). Between the grouping stage and the classifying stage, a sub-stage can be shown, which is responsible for calculating group features. In a system implementing the proposed method, the processing may be performed by hardware, firmware and/or software units incorporated in its sonar (or radar) receiver or in a remote processing unit (see FIG. 1a).

In order to reduce the computational resources and to exclude some of the noise, only strong echoes will be selected according to their received Signal to Noise Ratio (SNR) value, using a Detection Threshold (DT), which determines the size (intensity) of the detected object and the noise tolerance of the system.

The flow chart diagram of FIG. 2 utilizes the following main properties, features, and prior information.

### Echo Properties

Main echo properties characterize echoes received from a plurality of objects exposed to sonar or radar signals.

There are several main echo properties which the Inventors propose to use in the proposed method.

One or more echo properties taken for an echo “k” at time instance “m”, can be derived in the method. The echo properties list comprises at least τₖ^{m}, Iₖ^{m}, ρ_{k,l}^{m}, ρₖ^{m,m+1}
where
   τₖ^{m} is k'th echo's delay at time instance m
   Iₖ^{m} is k'th echo intensity at time instance m
   ρ_{k,l}^{m}, is the cross-correlation coefficient between the k, and l echoes' shapes, at time instance m
   ρₖ^{m,m+1} is the auto-correlation coefficient between the k'th echoes' shapes, at time instances m, and m+1.

As shown in the exemplary flow chart diagram of FIG. 2, the input of the Tracking stage 20 is formed by Echo properties (21) and by Prior information (marked 23), used as control information. Prior Information comprises at least accuracy and sensitivity data, and target's features, for example static and dynamic objects' characteristics or constraints, such as a constraint of continuity of movement of a human body (other constraints of dynamic objects kinematics can be used).

A human body can be virtually separated to a number of body parts (BPs). Each body part has its different kinematic pattern in different activities. This pattern can be captured by the body part displacement over time. The kinematic features of the human can be derived from the all group of body part displacements. Each body can be divided to relatively static components (e.g. torso, head), and to dynamic ones (e.g. upper and lower limbs moving while walking) components. Specific methods are known in the art for formulating so-called “sonar signatures” of human body and its parts.

In some cases, it is more informative to use not the absolute body part displacement, but the displacements relative to the torso. In activities like gait, where the whole body moves, some of the body parts, like the head, will have a relatively constant displacement from the torso, while the upper and lower limbs will have their periodic displacement patterns.

In case the main concern is tracking the human motion, the use of prior information comprising kinematic constraints can improve the tracking process accuracy. For example, the simple range of spread of different body parts can eliminate echoes, that are not related to the human. The control may also be provided by adjusting tracking criteria α,β,γ and parameters of the Branch metrics (will be described with reference to FIGS. 4a, b).

In the specific proposed diagram of FIG. 2, one may note that prior information/knowledge on targets (objects of interest) is utilized at each stage of the method. It should be emphasized, that usual prior art processing techniques utilize such information at the classification stage.

For reducing complexity of computation at the classification stage and increasing the accuracy of motion acquisition, the proposed method recommends using the prior info on targets' characteristic features before the classification stage (for example, in the grouping stage, in the tracking stage or both in the grouping and the tracking stages).

The functionality the tracking unit (stage) 20 will be briefly described with reference to FIGS. 3, 4.

The tracking stage/unit 20 produces, at its output 25, object properties per object, presented as sets of properties for respective detected and tracked objects. The object properties are determined by mapping echo properties to supposed objects.

One or more object properties taken for object n at time instance m, can be determined from the proposed list comprising at least
(dₙ^{m}, νₙ^{m}, Sₙ^{m}, Pₙ^{m}), where
   dₙ^{m}—is the n'th object location estimate at time instance m;
   νₙ^{m}—is the n'th object velocity estimate at time instance m, derived by either deviation of location estimates, dₙ^{m}, or by the Doppler effect;
   Sₙ^{m}—is the n'th object size estimate at time instance m, estimated for example by the number of echoes related to object n and their intensity
   Pₙ^{m}—is the n'th object pattern, for example estimated using spatial-temporal autocorrelation between echoes associated with object n.

In other words, the above object properties 25 can be understood as:

The distance (d) between the target and the sonar system at time instance m, Cₖⁿ, is the round trip time divided by factor of two. The distance covered by the sonar system is determined by the pulse repetition frequency and by the pulse width τ_{ρ},

Object position (location) can be obtained by range (distance) and azimuth and elevation angles from the sonar to the object, or by using multiple sonar sensors located at different locations, using statistical or geometrical methods.

Object velocity (ν) is a simple motion feature of the object, and can be obtained either by measuring of Doppler shift, or in case of high SNR of the system, by deviation of the object location.

The spatial-temporal correlations (proportional to P) of the different received echoes can indicate, in some cases, a characteristic of the object. In particular, —whether the echoes are related to the same or different objects, or are just interference.

Object dimensions (proportional to S) can be estimated by analysis of the number of echoes reflected from an object, their spatial spread, and by their energy. An indication to the object dimension is the echo's intensity, which is normalized by the factor of the range, and captures most of the echoes reflected from one proximate.

The object properties 25 are forwarded for grouping at the grouping stage (block 22) and form its first input.

The second control input 27 to block 22 is formed by Prior knowledge comprising the target's (for example, human body) characteristic features and/or constraints. The human body characteristics/constraints may include, for example. velocity/acceleration range, dimensions, sonar signature (pattern), etc.

It should be noted that the control box 27 takes into account at lest the target's class features/constraints, but preferably—those of the target's type (say, an elderly patient) and further preferably—of the type of implementation of the method (for example, watching an elderly patient when moving in a room). The grouping stage/block 22 can be divided into sub-stage of grouping objects based on similar properties (block 26), followed by or performed simultaneously with a sub-stage of determining main objects and sub-objects in the group (the hierarchy block 28). The Prior knowledge 27 is preferably used for the sub-stage 28, to determine main objects and sub-objects in the group. However, it may be also utilized for the grouping itself (26).

The grouping process may terminate by determining groups of object properties, wherein each group comprises sets of “object properties” of respective objects being members of the group. This result is shown as group properties (better to be called combined sets of object properties), per group, at the output 29 of the grouping block 22. The combined set of object properties of all members of the i'th group, at time instance m can be written down as follows:
{dₙ^{m,G}^{ᵢ}, νₙ^{m,G}^{ᵢ}, Sₙ^{m,G}^{ᵢ}, Pₙ^{m,G}^{ᵢ} . . . dₙ₊₁^{m,G}^{_{i . . .}} };

However, the grouping process may additionally comprise a step which is schematically shown as block 30. (Block 30 may be considered part of the grouping stage 22, but may be considered part of the classification stage 24).

Block 30 performs calculation of group features, per group, based on the combined sets of object properties of members of the group and the hierarchy of objects with a group. Each set of group features preferably comprises one or more of the following features derived for each i'th group over a time window “W”:ν^{Gi}, σ^{Gi}, and N^{Gi}, μ^{Gi}, σ_{ρ}^{Gi}

where:
ν^{Gi}—is average velocity in the group over a time window W, for instance, velocities may be relative to that of one moving or static object in the i'th group,
σ^{Gi}—is average location standard deviation over the time window W, possibly relative to one static or moving object in the i'th group,
N^{Gi}—is the number of dynamic/static objects in the i'th group over a time window W
μ_{ρ}^{Gi}—is average auto-correlation of the objects in the i'th group, and
σ_{ρ}^{Gi}—is standard deviation of the auto-correlation of the objects in the i'th group.

Alternatively to some of the above features, or in addition, some other group features may be determined, for example:

distribution between max and min velocities in the group, pattern similarities of objects in the group.

The sets of group features are fed to the classification stage processing 24.

The classification is performed under control of prior knowledge target features 31 or criteria 33 which actually implicitly incorporate the characteristic features and/or constraints of a supposed target (i.e., of the supposed target's class, and type, possibly of the type of implementation), Applying the control/criteria of 31, 33 enables to distinguish between dynamic-static or human-non-human character of the group. These features and criteria may be characteristics or thresholds of: velocity, standard deviation of location, spatial and temporal correlation and auto-correlation of echoes. Actually, the nature of the constraints 31 and criteria 33 respectively correspond to main group features mentioned above, so as to classify the groups according to their features and thus to distinguish the target.

The classification block 24 performs multi-level classification over the group features. As a result, a list of classes is produced, the groups are classified into static, dynamic, human and non-human classes. The human groups may be further classified by level and type of activity, so that even specific motions such as intruder's manipulations or falling/slipping of elderly patients may be recognized.

Therefore, if the target indeed belonged to one of those cases, it could be effectively distinguished and recognized as a result of the proposed grouping and classification procedure.

It should be noted that the diagram of FIG. 2 may be provided with a feedback connection between the outputs of the classification block 24 and the control blocks 23, 27, 31, 33, so as to adjust the prior knowledge according to the obtained classification results. Presence of the feedback would speak for a possibility of training the system. However, the proposed technology/system is effective even without any feedback/the training. The inventive technology is workable and capable of distinguishing targets based on at least prior knowledge on class/type of the supposed target, and does not require prior information about exact specific targets.

FIGS. 3a, 3b schematically show an exemplary processing approach used by the Inventors for tracking the detected objects.

The tracking stage can be performed as a dynamic, multiple object-, tracking by using various statistical criterions. For instance a Maximum Likelihood Estimator (MLE), implemented by Viterbi method, can be used for processing echo properties using a-prior information on the target of interest.

The tracking stage comprises detection (or so-called “creation” of echoes being supposed objects), deletion “ignoring” of echoes/object(s), splitting and/or merging of the created echoes/objects based on the selected statistical criterion. For performing these operations, the echo properties (of location, intensity and correlation) are necessary, as well as the prior information discussed above.

The multi-object dynamic tracking may be performed as the orthogonal low complexity (and thus efficient) approximation to the recursive maximum likelihood estimator MLE, with said constraints typical to desired target's motion.

The mentioned constraints relate to the target's expected displacement, intensity and correlation patterns—i.e., the constraints being a priori information.

In a specific version of the method, a trellis diagram (FIG. 3a) is utilized for implementation of the tracking algorithm. The states of the diagram are the set of the distance (range) between the object and the sonar of the detected echoes. In the upper part, the branch metric with the lowest value (M₁₁) over the constraint length (4 pulse repetitions in this example) is chosen and maximizes the MLE criterion. The algorithm is capable of mitigating for misdetection, as in the lower figure, by interpolation, and to dynamically delete and create new objects without prior assumptions.

The exemplary approach uses a sequential Maximum Likelihood Estimator (MLE) with metrics that correspond to the displacements, intensity, and pattern constraints of a human body. The sequential MLE for object detection can be implemented by using algorithms similar to the Viterbi algorithm. It includes maximization of the object probability function at a certain location using constraints based on continuity of the motion.

The trellis diagram of FIG. 4a represents different locations of the objects to be tracked. For M discrete locations, states at time instance m, (Sₖ^{m} and Sₗ^{m}), represent the location of the k'th and l'th echoes (supposed objects). A path in the diagram is a transition between states at consecutive discrete time intervals. Each possible transition represents a possible motion of the object from one position to another. The transition between the states depends on the Pulse Repetition Rate (PRF), and on the motion. Slow motion with high PRF will have fewer transitions in the trellis diagram.

In FIG. 3a, objects tracked/presented as paths/branches B, C respectively including states S2 and S3 (shown as darker ovals) are considered not related to the object tracked/presented as path/branch A including state S1. The metric parameters M₁₂ and M₁₃ of B and C do not correspond to M₁₁ of the A and thus they are not merged). The term “metric” will be explained below. The result may be such that the two lower branches B and C are considered to belong to one, dynamic object, while branch A—to another, static object. FIG. 3b illustrates intensities of the objects (i.e., of their echoes) over the same time, in the coordinates of time and space.

Each legal transition between states at time instance m, can be defined as a branch with a branch metric M_{k,l}^{m}, which is a function of the similarity between consecutive states. The novel, proposed by the Inventors Branch Metric will be further described.

Any statistic procedure comprises a correlation sub-step for making a decision whether an object with a specific tracked behavior is associated with another tracked object (and thus should be further tracked in parallel, or dropped, or merged); there is a need in a correlation tool/measure.

For use in the statistical tracking, the Inventors have proposed a simplified Branch Metric Approximation, as a correlation tool. Metrics used in the prior art are quite complex and result in heavy computations. The proposed branch metric is a function of the distance between two states, the pattern of the echo, and the echo intensity.

As mentioned in the Summary by expression (1), the branch metric between object k, and l, can be defined close to:

M_{k,l}^{m}=e^{-aΔD}^{_{k,l}}^{^{m}}(I_{k,l}^{m})^{β}(ρ_{k,l}^{m})^{γ}, (1)

where

$$Δ {d}_{k , l}^{m} = \vert {d}_{k}^{m} - {d}_{l}^{m - 1} \vert , {I}_{k , l}^{m} = \frac{min ( {I}_{k}^{m} , {I}_{l}^{m - 1} )}{max ( {I}_{k}^{m} , {I}_{l}^{m - 1} )} ,$$

ρ_{k,l}^{m}, are measures of distance, intensity, and cross-correlation between the k'th and the l'th echoes (supposed objects), and

α, β, γ, are constants determined experimentally and reflecting reliability and significance of the distance, the intensity, and the correlation measures respectively to the detection probability.

The indexes in the Metrics are indexes of the echoes being supposed objects. Only at the output of the tracking procedure the indexes become indexes of the objects.

The branch metric can be normalized to values between 0 and 1 to represent a probability function.

An object i'th path metric is the sum of the branch metrics that are related to the objects in the time interval w:

Cᵢ^{m}=Σ_{m′=m-W+1}^{m′=m}M_{i,j}^{m′}. (3)

The metrics M_{i,j}^{m′} used in the above expression is usually a complex statistical measure. It may be now replaced with the one (M_{k,l}^{m}) simplified by the Inventors and stated in (1).

The time interval W is called a constraint length and must be big enough to reflect sufficient statistics to detect the object. The too long constraint length would summarize noise and affect the tracking of fast movements. And vice versa, the too short constraint may be useless for detecting slow movements.

An object j at instance time m, is selected to be related to an object i, according to the following criterion which can be implemented by Viterbi method:

j=argmaxⱼ,(Cᵢ^{m-1}+M_{i,j′}^{m}). (4)

Whenever there is a change in the medium, an object can be created, merged with an existing one, or deleted from the trellis diagram. This enables flexibility of the tracking scheme, and tracking of dynamic objects, that can come or exit the range of the sonar, and change their properties. The extension can include: object splitting, creations, deletion, and interpolation. Exemplary scenarios are as follows:
1) Object Splitting: in case of a movement of a body part out of the torso, like lifting the arm, a new object that relate to torso will be created. In the trellis diagram, the new object is seen as a split of the branch metric of previous object.
2) Object Creation: in case new object approaches the sonar coverage range, e.g. a new person enters the room, a new object is created. If there is no other object in the trellis diagram with close enough metric to the new one, and the object exists for over certain duration, usually in the range of the constraint length, an object is created in the diagram.
3) Object Merging: in case two objects with similar properties, approach each other, like a person carrying a bag, the two related proximate objects in the trellis diagram will coincide to one.
4) Object Deletion: in case an object leaves the sonar coverage range, or gets far from the sonar and intensity goes below the detection threshold, the object path is cut in the trellis diagram.
5) Object Interpolation: is performed to mitigate over missing estimations of an object due to noise, or scatterers, along the constraint length W.

As a result of the tracking algorithm, the basic echo properties are processed together with the prior information constraints.

The tracking stage terminates with obtaining respective sets of object properties for all objects being tracked, over time (which means, each object property as a sequence of its values at time instances “m”). Due to the broad band sonar signal used in the system, accuracy of the obtained properties are quite high.

FIGS. 4(a, b, c, d, e, f) show experimentally obtained measurements which illustrate efficiency of the grouping stage performed upon the tracking stage according to the proposed method.

The stage of grouping objects (to groups or clusters) is preferably performed as an unsupervised segmentation process, i.e., without any previous training of the system. The grouping starts with receiving object properties, for all of the tracked objects.

The grouping is based on similar properties between objects of the same group, for example on the location property—by using a simple proximity principle which may also be formulated as spatial and temporal correlation between objects' reflections.

The grouping stage further comprises selecting, per group, a main object and sub-objects thereof, based on a-priori knowledge about the objects being tracked, such as prior knowledge on kinematics of dynamic objects of interest (humans/animals, etc.).

The grouping stage may comprise dividing the sub-objects into static and dynamic objects using, for example, a threshold of standard deviation of the sub-object(s) location from the main object(s).

The grouping stage may utilize a graph model, by building a graph being a model of connections between objects represented by intensities of their reflections/echoes, and by further braking connections which do not demonstrate dependence/correlation of objects to one another in the currently used constraint/window Time-Distance (TD).

The criteria may be as follows. One group should have one main object and one or more sub-objects which have less intensity than the main object. If the sub-objects are connected/correlated with one another in the graph and have similar intensities, connections there-between in the graph may be broken. Generally, if a connection/correlation to another “main” object appears, such a connection should also be broken, based on a predetermined quantitative criterion, to make the sub-object grouped with only one main object. Alternative hierarchies may be used instead of the proposed one.

The Inventors have also proposed that the time-distance criterion—the TD constraint—be selected for the grouping, according to the character of objects to be grouped while tracked (for example, a greater distance, smaller “time” window for a moving sportsman; a smaller “distance” longer “time” window for a sick person to be monitored) Time interval T may be called a constraint length.

FIGS. 4 a-f are time-space sonar diagrams which generally illustrate results of tracking and further grouping of the tracked objects according to the proposed invention. FIG. 4.a shows results of the matched filter, and the threshold operation before the tracking stage, i.e. only strong echoes with relevant frequencies are considered. FIG. 4.b shows results of applying the sequential MLE object tracking, where more than 20 different objects were created (they are shown by different colors appearing with quite weak regularity at different lines of the graph) FIG. 4.c shows the first stage of the post processing in which missing estimations are mitigated for each object by interpolation; the picture has become more clear. Then the grouping operation is performed on the objects. First, the main object is detected in the space-time diagram. Then its related sub objects, with lower intensity, are chosen. Intermediate results of the grouping are shown in FIG. 4.d, where each group has its different color. Intermediate groups are formed by: the black lines group at the bottom of the drawing are supposed to be sonar itself, the lower blue sinus-like line is supposed to be a human, the upper green sinus-like line and the green straight line are supposed to be a wall, and the uppermost group of lines of different colors supposed to belong to the wall or the nearby furniture. Grouping errors, like in the middle of FIG. 4.d where the wall (in green) is merged with the nearby human and erroneously shown as a sinusoidal green line, can be minimized by using the merge and split algorithm based on a similarity measure of different features of the groups. FIG. 4.e shows the results after the group merging and splitting where the person's objects (say, the body, the head and the hands) belong to the person's group (marked H, with the two sinusoidal lines, now both being blue). Other groups are marked WL1, WL2 for the wall and objects on it, and So for the sonar equipment. In this example, to derive the feature of number of dynamic objects in the group, each object in a group is sorted to a dynamic or static object according to their standard deviation from the main object. FIG. 4.f shows the results of this process: for the group of a human, the main object (torso) is seen as the red line in the middle, being a thick sinusoidal line and marked H main); its related static object (head) is seen as a black thin line accompanying the main sinusoid, and its related dynamic objects (legs, hands) are seen ad purple lines looking like protuberances near the black thin line. Main objects of other groups are seen as other three red (thick) lines at the lower and upper portions of the drawing (WL1main, WL2main, So-main).

The reference is now made to FIGS. 5 a-d which show a specific example for representation of the grouping by schematic graphs. The example of FIGS. 5 a-d illustrates a simple suboptimal grouping scheme for the criterion that is based on spatio-temporal and intensity properties of the objects. It includes four sub-stages.

In sub-stage a, the objects' location (7 objects are seen) is just a state diagram.

In sub-stage b, objects that reside in a pre-determined object range (for human, it is around 0.5 m, which is the human limbs' maximal span) are connected according to the similar intensity and proximity parameters. The direction of connections can be chosen according to duration of the objects' presence in the diagram. Alternatively, the direction of connections may be selected from higher intensity to lower intensity, etc.

In stage c, connections of objects, having short duration, with other objects are disconnected, using a likelihood criterion around the local object with the maximal intensity. Connections between objects having similar low intensities may be disconnected as well. In the example, three connections have been disconnected.

Then in a final sub-stage d, main objects and sub-objects are selected. The objects with the longer duration and most intense objects are chosen as the main objects in their groups, and other objects connected to the main objects in their groups, —as sub objects.

In some circumstances, a group can be disrupted by other objects in the medium. As a result, some or all of the group's objects can be merged with a different cluster/group. For example, when a human approaches a wall, the respective groups that relate to the human and the wall may be merged by mistake (see FIG. 4d). In these cases, different groups need to be merged (to fix the case of disruption), or to be split (to fix the wrong merger with another group). Algorithms for objects merging and splitting can be based on a similarity measure of various features of the groups, like standard deviation, velocity and distance between different groups.

The merging and the splitting operations for groups may be based on a statistical (say, MLE) similarity measure of various features (for example, of the prior knowledge about target characteristic features or constraints), to minimize wrong assignment of objects.

FIG. 6 shows a schematic diagram for the optional sorting (categorizing) of objects in the group to static and dynamic. The operation may still be performed at the grouping stage.

For further assessment of activity, the objects can be divided to additional categories, according to their size, location, and kinematics statistics. A fundamental category is of dynamic and static objects. Dynamic objects are sub-objects that fluctuate more than a certain threshold, usually around the main body of the group, e.g. lower and upper limbs are dynamic parts, while walking. Static objects are sub-objects that are relatively static in relation to the main-body (torso), e.g. the head. A threshold on the standard deviation of the object location from the main object location can be used to determine if an object is dynamic or static. FIG. 6 illustrates a running human, and shows its related objects being members of one group.

At the end of the grouping stage, calculation of the group features is performed, which has been described with reference to Block 30 of FIG. 2.

FIGS. 7a, 7b, 7c, 7d are examples demonstrating effect of applying prior knowledge constraints at the grouping stage of processing object properties. A first experiment (FIGS. 7a, 7b) shows the effect of applying a constraint of location range from the main object in the group. A second experiment (FIGS. 7c, 7d), shows the influence of a constraint of continuity of human movement.

The first experiment included a standing human, a chair and a wall exposed to signals transmitted by a sonar transmitter located at the opposite wall of the indoor environment (a room).

FIG. 7a shows the grouping results for the case where the location range constraint was selected as 0.6 m which corresponds to possible location range of human hands or legs relatively to the human torso. The result of grouping is satisfactory: the uppermost group of red lines demonstrate less than 0.6 location range and are mapped to the wall group WL; the second from the top group of green lines demonstrate the range of about 0.6 m, and are thus mapped to the human body group (the second group H from above), the blue lines are mapped to the chair group (the group Ch of three straight lines, with a very small location range), and the lowest group of black lines are a so-called self-sonar group So formed by self-echoes of the sonar device,

FIG. 7b shows the case where the location range constraint was erroneously selected as 1.2 m, i.e. actually it did not characterize a human body.

The grouping result in this case was therefore erroneous and comprised only two groups, namely the wall and the human were mapped together into the “brown” group WL+H (all the lines in the upper half of the drawing), while the chair and the sonar were mapped in a common “blue” group Ch+So (all the lines in the lower half of the drawing).

The second experiment included two persons asynchronously crossing the room to and from the sonar transmitter.

FIG. 7c shows the case of applying the constraint of movement continuity (corresponding to continuity in the direction of velocity, which is typical to humans). The grouping result was good, i.e. the two persons, presented by two sinusoidal lines shifted by phase, were distinguished even at points where they almost met in the room. The two sinusoidal trajectories of different colors/persons correspond to two different groups H1, H2, each group comprising group members formed by the human torso, legs and hands presented by lines accompanying the two respective sinusoidal lines.

FIG. 7d illustrates the case where the movement continuity constraint was not applied and, as a result, two erroneous groups H1, H2 (in the form of zig-zags) were formed and even an additional erroneous group H3 appeared, which incorrectly represent the movement of the two humans.

FIG. 8 shows a schematic diagram of a specific example of the proposed classification stage—i.e., for the Human Motion Classification.

The Classifying stage of the method performs classification of all the objects grouped to all different groups by applying, to all said objects in the groups, prior knowledge about features of objects of interest (e.g. knowledge of human kinematics) and suitable criteria to recognize static, dynamic, human and non-human objects for further classification of more detailed motion activity of the human objects.

The classification stage, in general, may be performed by using conventional motion classification methods. However, in the proposed method the classification stage is non-standard at least due to the fact it is based on the preceding, novel grouping stage, i.e. on the set of group features produced at the groping stage. The grouping stage is preferably performed without any pre-classification.

The classification is preferably performed in a multi-level way, for example by a two level decision tree k-NN classifier for activity type and activity level estimation.

The classification is performed by comparing the group features with the prior knowledge about the targets' classes and types, possibly/preferably also on the expected/predetermined implementation type, and/or with criteria (such as thresholds and ranges) derived from the prior knowledge.

In FIG. 8, three types of classifiers are shown in a two-level decision tree algorithm for activity type and activity level estimation, according to which groups of objects are first divided into static and dynamic (the first classifier 40), and then static groups are further classified to human and non-human (the second classifier 42); while dynamic groups of objects which are presumably human are further classified by activity type and activity level (third classifier 44). Static groups of human objects may be then “forwarded” to the 3^{rd} classifier 44 and further classified by activity type and activity level.

The classification may be performed as “over-time” classification (for example, for complicate types of movement).

Human motion classification can be performed using such a feature of a possible target as its sonar signature. Different objects, and different people, and people with different activity types, have different sonar signatures. In sonar system, reflections are mostly from the body surface] and therefore, human detection can be obtained by its unique body surface structure that is different from other objects. In addition, the kinematic features that are unique to humans, can be aggregated.

One of the main advantages of the proposed technique is that the stages of tracking, and grouping do not perform pre-classifying but reduce complexity of the classifications stage, so that no pre-classifying or training is required in advance.

FIG. 9 shows an example showing how classification results of activity type are demonstrated by a so-called feature-space diagram formed in three axes of: 1) velocity, 2) Number of objects and 3) standard deviation, all being the group features.

The figure allows seeing static and standing objects (blue squares and black crosses) in a crowded lowermost cluster near one another), walking human objects as green rings highly dispersed over the space, and objects with the swinging motion of hands—as red romboids which are moderately dispersed from the crowded cluster.

For the activity classifier, it seems that the velocity feature is the most significant one in case of activity type that involves walking, while the feature of standard deviation is the more significant to distinguish between swinging hands and standing.

Other mentioned classifier types could be demonstrated in modified feature space diagrams, that would show that upon the proposed tracking and grouping, the objects exposed to sonar/radar signals can be successfully separable in the different feature spaces for the three decision tree classifiers. The use of relatively simple classifier like the k-NN classifier is justified, as giving an efficient reasonable performance, as it can operate well with separable distributions with a relatively low complexity.

While the invention has been described with reference to specific examples only, it should be appreciated that other versions of the method can be suggested based on the described principles and options, that various embodiments of suitable sonar/radar systems can be designed for implementing the disclosed technology, and that such versions and embodiments should be considered part of the present invention whenever covered by the claims which to follow.

### LIST OF NON-PATENT REFERENCES

1. S. S. Ram, Y. Li, A. Lin, and H. Ling, “Doppler-based detection and tracking of humans in indoor environments,” Journal of the Franklin Institute, vol. 345, pp. 679-699, 2008.
2. G. Blumrosen, B. Hod, T. Anker, B. Rubinsky, and D. Dolev, “Enhancing RSSI-based Tracking Accuracy in Wireless Sensor
3. J. M. Hausdorff, M. E. Cudkowicz, R. Firtion, J. Y. Wei, and A. L. Goldberger, “Gait variability and basal ganglia disorders: stride-to-stride variations of gait cycle timing in Parkinson's disease and Huntington's disease,” Mov Disord, vol. 13, pp. 428-437, 1998.
4. G. Blumrosen, M. Uziel, B. Rubinsky, and D. Porrat, “Noncontact tremor characterization using low-power wideband radar technology,” IEEE Trans Biomed Eng, vol. 59, pp. 674-686, 2012.
5. E. M. Staderini, “UWB radars in medicine,” Aerospace and Electronic Systems Magazine, IEEE, vol. 17, pp. 13-18, 2002.
6. G. Blumrosen, B. Fishman, and Y. Yovel, “Non-contact Ultra-Wideband Sonar

1. A method for distinguishing a target, wherein the target including one or more objects of interest possibly located among a plurality of objects, the method comprising stages of:
obtaining and processing sonar or radar raw data,
tracking the objects of the plurality using the processed raw data,
grouping the tracked objects by associating them into one or more groups, while hierarchically arranging the tracked objects in the groups and controllably applying prior knowledge at least about characteristic features and/or constraints of the target's class;
classifying the groups to classes and determining whether any of the groups matches to the target's class.

2. The method according to claim 1, wherein the controlled applying of the prior knowledge at the grouping stage comprises applying additional features and/or constraints being characteristic for a specific type of the target and for a specific type of the method implementation.

3. The method according to claim 1, wherein said features or said constraints are selected from the following non-exhaustive list: dimensions, intensity, sonar/radar signatures of the target's class or type, acceleration range, velocity range, reflection structure, pattern of change, time-distance constraint TD.

4. The method according to claim 1, comprising a preliminary stage of receiving echoes of the signals from the objects and deriving the raw data from said echoes,
the method further comprising.
performing the raw data processing, thereby obtaining one or more echo properties taken for an echo “k” at time instance “m”, the one or more echo properties being selected from an echo properties list comprising at least (τₖ^{m}, Iₖ^{m}, ρ_{k,l}^{m}, ρₖ^{m,m+1})
where
τₖ^{m} is k'th echo's delay at time instance m
Iₖ^{m} is k'th echo intensity at time instance m
ρ_{k,l}^{m}, is the cross-correlation coefficient between the k, and l echoes' shapes, at time instance m
ρₖ^{m,m+1} is the auto-correlation coefficient between the k'th echoes' shapes, at time instances m, and m+1.
performing the stage of tracking the objects, comprising controlled processing of the one or more obtained echo properties and mapping thereof to objects, thereby obtaining, for each of the objects, one or more object properties taken for object n at time instance m, the one or more object properties being selected from an object properties list comprising at least
(dₙ^{m}, νₙ^{m}, Sₙ^{m}, Pₙ^{m}), where
dₙ^{m}—is the n'th object location estimate at time instance m;
νₙ^{m}—is the n'th object velocity estimate at time instance m;
Sₙ^{m}—is the n'th object size estimate at time instance m;
Pₙ^{m}—is the n'th object pattern;
performing the stage of grouping of the objects by controllably associating the objects into groups based on one or more similar said object properties, with the hierarchically arrangement of the objects being members in the groups so that each of the groups comprises a main object and at least one sub-object, thereby obtaining for each of the groups a combined set of object properties of all members of the i'th group, at time instance m, being
{dₙ^{m,G}^{ᵢ}, νₙ^{m,G}^{ᵢ}, Sₙ^{m,G}^{ᵢ}, Pₙ^{m,G}^{ᵢ} . . . dₙ₊₁^{m,G}^{ᵢ} . . . };
based on the combined set of object properties and their hierarchy, deriving one or more group features, for each i'th group over a time window “W”, the group features being selected from a group features list comprising at least (ν^{Gi},σ^{Gi}, N^{Gi}, μ_{ρ}^{Gi}, σ_{ρ}^{Gi}), where
ν^{Gi}—is average velocity in the group over a time window W,
σ^{Gi}—is average location standard deviation over the time window W,
N^{Gi}—is the number of dynamic/static objects in the i'th group over the time window W,
μ_{ρ}^{Gi}—is average auto-correlation of the objects in the i'th group,
σ_{ρ}^{Gi}—is standard deviation of the auto-correlation of the objects in the i'th group;
performing the classification stage by controllably applying, to the group features per group, at least prior knowledge on characterizing features and/or constraints of the target's class, corresponding to one or more of the group features, thereby determining whether at least one of the groups matches to the target's class.

5. The method according to claim 1, wherein the stage of tracking is performed flexibly and controllably, with
applying prior information about the target's class, and
performing splitting and/or merging of the echoes for tracking newly appearing, disappearing, and/or transforming objects in the plurality.

6. The method according to claim 1, wherein the tracking stage is performed as a statistical similarity tracking procedure, using a statistical criterion such Maximum Likelihood Estimator MLE, Minimal Mean Square Error MMSE, or the like.

7. The method according to claim 6 utilizing, for the statistical similarity tracking procedure, a correlation tool being a simplified Branch Metric,
wherein the simplified Branch Metric M_{k,l}^{m} between echoes k and l for time instance m, is determined substantially close to:
M_{k,l}^{m}=e^{-aΔD}^{_{k,l}}^{^{m}}(I_{k,l}^{m})^{β}(ρ_{k,l}^{m})^{γ}, (1)
and where
$$Δ {d}_{k , l}^{m} = \vert {d}_{k}^{m} - {d}_{l}^{m - 1} \vert , {I}_{k , l}^{m} = \frac{min ( {I}_{k}^{m} , {I}_{l}^{m - 1} )}{max ( {I}_{k}^{m} , {I}_{l}^{m - 1} )} ,$$
ρ_{k,l}^{m}, are measures of distance d, intensity I, and cross-correlation ρ between the k'th and the l'th echoes, and α, β, γ, are constants.

8. The method according to claim 1, wherein
the stage of grouping comprises:
receiving object properties of each of the objects, upon being determined at the tracking stage;
associating the objects into groups by utilizing the received object properties, so that each of the groups is formed based on statistical similarity of at least one of the object properties for members of the group; thereby presenting each group as a combined set of object properties of all members of the group;
simultaneously with, or after the association of the objects into groups, hierarchically arranging the objects in each of the groups by controllably applying the prior knowledge at least in the form of one or more features or constraints characteristic for the target's class.

9. The method according to claim 8, further comprising providing control in the grouping stage by selecting the object property for forming groups and/or by selecting the prior knowledge in the form of constraints related to types of objects of interest and according to specific implementations of the method.

10. The method according to claim 1, wherein the grouping stage comprises iterative procedures of merging and/or splitting the formed groups.

11. The method according to claim 1, wherein the classification stage comprises:
obtaining group features derived for each of the groups, and
based on the group features and on prior knowledge on different classes of targets, forming a list of classes for the groups, determining the class of each group, wherein the classes comprising at least static/dynamic and human/non-human classes, and further determining type, activity type and level for at least some of the groups.

12. The method according to claim 1, comprising
a preliminary step of exposing the plurality of objects to the signals, including:
emitting a combination of sonar signals comprising more than one predetermined different frequencies in a bandwidth between of about 5 kHz and of about 1000 kHz;
applying the combination of the sonar signals to an area where the plurality of objects including one or more targets are expected to be located.

13. The method according to claim 1, adapted for distinguishing the target among said plurality of objects in an underwater environment.

14. A system for distinguishing a target, the target including one or more objects of interest possibly located among a plurality of objects exposed to sonar or radar signals,
the system comprising a processing block accommodating therein:
a unit for processing sonar or radar raw data obtained from the plurality of objects,
a unit for tracking the objects using the processed raw data,
a unit for grouping the tracked objects by associating the objects into groups while hierarchically arranging the tracked objects in the groups, wherein the unit for grouping is controlled at least by applying prior knowledge about characteristic features or constraints of the target's class;
a unit for classifying the groups and recognizing the target by matching the groups classes to the target's class.

15. The system according to claim 14, further comprising a transmitter for transmitting the sonar or radar signals, a receiver for detecting echoes thereof and extracting raw data from the echoes, a synchronizing means between the receiver and the transmitter, and a communication line for forwarding the raw data to the processing block.

16. The system according to claim 14, further comprising a sonar or a radar assembly configured for creating image of the target based on results produced by said processing block.

17. The system according to claim 17, wherein said assembly is an ultrasound device adapted for creating images of internal body parts of a patient.

18. The system according to claim 14, designed for distinguishing the target among said plurality of objects in an underwater environment.

19. A software product comprising computer implementable instructions and/or data for carrying out the method according to claim 1, the software product being stored on an appropriate computer readable storage medium so that the software is capable of enabling operations of said method when used in a computerized system.

20. A computer readable storage medium accommodating the software product according to claim 19, or a portion thereof.
